(19) United States
(12) Patent Application Publication
(10) Pub. No.: US 2026/0174869 A1
(43) Pub. Date: Jun. 25, 2026

(54) PROTEIN POWDER COMPOSITIONS AND METHODS FOR PREPARING THE SAME

(71) Applicant: Applied Materials, Inc., Santa Clara, CA (US)

(72) Inventors: Fei WANG, Fremont, CA (US); Kedar Anand SAPRE, San Jose, CA (US); Suneel RASTOGI, Hillsborough, NJ (US); Shivkumar CHIRUVOLU, San Jose, CA (US)

(21) Appl. No.: 19/124,245

(22) PCT Filed: Oct. 12, 2023
(86) PCT No.: PCT/US2023/035019

§ 371 Date: Apr. 24, 2025

Related U.S. Application Data
(60) Provisional application No. 63/420,377, filed on Oct. 28, 2022.

(51) Int. Cl. A61K 47/26; A61K 47/18; B22F 1/16; C23C 16/52

(52) U.S. Cl. CPC A61K 47/26; A61K 47/183; B22F 1/16; C23C 16/52; B22F 2998/10

(57) ABSTRACT

Embodiments of the present disclosure generally relate to protein powder compositions and methods for preparing the same. In one or more embodiments, methods include coating a plurality of protein powder particles containing myoglobin with an aluminum oxide coating to form a plurality of coated particles during an atomic layer coating (ALC) process. The protein powder compositions have improved properties over the corresponding uncoated protein powder. The protein powder compositions have greater flowability, density, and a greater level of recoverability over the corresponding uncoated protein powder.

### BACKGROUND

### Field

Embodiments of the present disclosure generally relate to protein powder compositions and methods for preparing protein powder compositions, and more specifically, coated protein powders and the coating processes for preparing the same.

### Description of the Related Art

Vaccines are generally protein based and can have a relatively short shelf-life even if maintained at temperatures less than ambient. The stability of proteins in solid state are prone to aggregation during storage and require formulation and refrigerated storage. Lyophilized and spray dried formulations can exhibit poor flowability. Majority solid protein formulation need refrigerated storage even after the use excipients to stabilize the formulation. There are formulation of protein stabilized by sugar molecular which can be stable for several days under ambient condition, but not robust practice.

Therefore, there is a need for improved protein powder compositions and method to prepare protein powder compositions.

### SUMMARY

Embodiments of the present disclosure generally relate methods for forming or otherwise preparing protein powder compositions as well as the compositions formed by such methods.

In one or more embodiments, a method of forming a protein powder composition is provided and includes positioning a plurality of protein powder particles within a process region of a processing chamber, and coating the plurality of protein powder particles with an aluminum oxide coating to form a plurality of coated particles during an atomic layer coating (ALC) process. Each of the protein powder particles comprises myoglobin. The ALC process includes one or more deposition cycles, and each of the deposition cycles includes exposing the plurality of protein powder particles to an aluminum precursor, infiltrating the plurality of protein powder particles with the aluminum precursor via spaces between the protein powder particles, purging the process region to remove gaseous remnants containing the aluminum precursor, exposing the plurality of protein powder particles to an oxidizing agent, infiltrating the plurality of protein powder particles with the oxidizing agent via spaces between the protein powder particles to produce the aluminum oxide coating disposed on outer surface of each of the protein powder particles, and purging the process region to remove gaseous remnants containing the oxidizing agent.

In some embodiments, a method of forming a protein powder composition is provided and includes positioning a plurality of protein powder particles within a process region of a processing chamber, where each of the protein powder particles comprises myoglobin. The method also includes coating the plurality of protein powder particles with an aluminum oxide coating to form a plurality of coated particles during an ALC process, where the plurality of coated particles has a greater flowability value than the plurality of protein powder particles. The ALC process includes one or more deposition cycles, and each of the deposition cycles includes exposing the plurality of protein powder particles to an aluminum precursor, purging the process region to remove gaseous remnants containing the aluminum precursor, exposing the plurality of protein powder particles to an oxidizing agent, and purging the process region to remove gaseous remnants containing the oxidizing agent.

In other embodiments, protein powder compositions are provided and contain a plurality of coated particles, where each of the coated particles contains a protein powder particle containing myoglobin and a coating containing aluminum oxide disposed around each of the protein powder particles.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a graph illustrating reconstitution time for a variety of samples of protein powders, as described and discussed in one or more embodiments herein.

FIG. 2 is a graph illustrating moisture concentration for a variety of samples of protein powders, as described and discussed in one or more embodiments herein.

FIG. 3 is a graph illustrating soluble protein recovery for samples of lyophilized protein powders, as described and discussed in one or more embodiments herein.

FIG. 4 is a graph illustrating soluble protein recovery for samples of unformulated protein powders, as described and discussed in one or more embodiments herein.

FIG. 5 is a graph illustrating soluble protein recovery for samples of spray dried protein powders, as described and discussed in one or more embodiments herein.

FIG. 6 is a graph illustrating the relative quantity of aggregates in soluble protein for samples of lyophilized protein powders, as described and discussed in one or more embodiments herein.

FIG. 7 is a graph illustrating the relative quantity of aggregates in soluble protein for samples of unformulated protein powders, as described and discussed in one or more embodiments herein.

FIG. 8 is a graph illustrating the relative quantity of aggregates in soluble protein for samples of spray dried protein powders, as described and discussed in one or more embodiments herein.

To facilitate understanding, identical reference numerals have been used, where possible, to designate identical elements that are common to the Figures. It is contemplated that elements and features of one or more embodiments may be beneficially incorporated in other embodiments.

### DETAILED DESCRIPTION

Embodiments of the present disclosure generally relate methods for forming or otherwise preparing protein powder compositions as well as the compositions formed by such methods. The protein powder compositions have improved properties over the corresponding uncoated protein powder. The protein powder compositions have greater flowability, density, and a greater level of recoverability over the corresponding uncoated protein powder. The protein powder compositions can be or include coated medical compositions or drugs including vaccines, active pharmaceutical ingredients (APIs), as well as various food and/or flavoring products. In some examples, the protein powder compositions can be or include a vaccine having a coating containing aluminum oxide or alumina which has an extended and/or more stable shelf-life than the same vaccine without the coating.

In one or more embodiments, methods for preparing or otherwise forming a protein powder composition are provided and include positioning a plurality of protein powder particles within a process region of a processing chamber. The method also includes coating the plurality of protein powder particles with an aluminum oxide coating to form a plurality of coated particles during an ALC process. The ALC process includes one or more deposition cycles, and each of the deposition cycles includes exposing the plurality of protein powder particles to an aluminum precursor, purging the process region to remove gaseous remnants containing the aluminum precursor, exposing the plurality of protein powder particles to an oxidizing agent, and purging the process region to remove gaseous remnants containing the oxidizing agent.

The protein powder composition contains a plurality of coated particles, where each of the coated particles has a protein powder particle containing at least myoglobin and a coating disposed around each of the protein powder particles. The coating contains aluminum oxide.

In one or more embodiment, the plurality of coated particles has a greater flowability value than the plurality of uncoated protein powder particles. In some embodiments, the plurality of coated particles has a greater bulk density than the plurality of protein powder particles.

The protein powder particles are a particulate form of a protein composition. The protein composition and/or the each of the protein powder particles contain, include, comprise, consists, or consist essentially of one or more proteins and optionally one or more other components. Exemplary proteins can be or include myoglobin, lysozyme, capsid, one or more polypeptides, derivatives thereof, or any combination thereof.

In some embodiments, the protein composition and/or the each of the protein powder particles contain, include, comprise, or consist essentially of one or more sugars and/or one or more sugar alcohols. Exemplary sugars and/or sugar alcohols can be or include sucrose, mannitol, lactose, trehalose, sorbitol, isomers thereof, derivatives thereof, salts thereof, or any combination thereof. The protein composition and/or the each of the protein powder particles contain about 0.01 wt % to about 15 wt % of the sugar.

In some embodiments, the protein composition and/or the each of the protein powder particles contain, include, or comprise of one or more buffers. Exemplary buffers can be or include potassium phosphate (e.g., potassium dihydrogen phosphate, dipotassium hydrogen phosphate, or a mixture thereof), sodium phosphate (e.g., sodium dihydrogen phosphate, disodium hydrogen phosphate, or a mixture thereof), derivatives thereof, or any combination thereof. The protein composition and/or the each of the protein powder particles contain about 0.01 wt % to about 15 wt % of the buffer.

In some embodiments, the protein composition and/or the each of the protein powder particles contain, include, comprise, or consist essentially of one or more additives and/or one or more other components. Exemplary additives and/or other components can be or include one or more surfactants, one or more isotonicity agents, one or more antioxidants, one or more chelators, one or more preservatives, one or more amino acids, one or more monomers, one or more polymers, derivatives thereof, or any combination thereof. The protein composition and/or the each of the protein powder particles contain about 0.01 wt % to about 15 wt % of the additive and/or other component.

The protein composition can be processed or otherwise transformed into particles to produce the protein powder particles by various techniques. In one or more examples, the plurality of protein powder particles are produced from a spray dried process. The protein composition is spray dried to produce the protein powder particles. In other examples, the plurality of protein powder particles are produced from a lyophilization process. The protein composition is lyophilized to produce the protein powder particles.

In one or more embodiments, the plurality of protein powder particles has an average particle size in a range from about 0.1 μm, about 0.5 μm, about 1 μm, about 2 μm, about 5 μm, about 8 μm, about 10 μm, about 15 μm, about 20 μm, about 30 μm, about 40 μm, about 50 μm, about 60 μm, about 80 μm, or about 100 μm to about 120 μm, about 150 μm, about 200 μm, about 250 μm, about 300 μm, about 400 μm, about 500 μm, about 600 μm, about 800 μm, about 800 μm, about 900 μm, about 1,000 μm, about 1,200 μm, about 1,500 μm, or greater. For example, the plurality of protein powder particles has an average particle size in a range from about 0.1 μm to about 1,500 μm, about 1 μm to about 1,500 μm, about 1 μm to about 1,200 μm, about 1 μm to about 1,000 μm, about 1 μm to about 800 μm, about 1 μm to about 600 μm, about 1 μm to about 500 μm, about 1 μm to about 400 μm, about 1 μm to about 300 μm, about 1 μm to about 200 μm, about 1 μm to about 150 μm, about 1 μm to about 100 μm, about 1 μm to about 80 μm, about 1 μm to about 50 μm, about 1 μm to about 35 μm, about 1 μm to about 20 μm, about 1 μm to about 15 μm, about 1 μm to about 10 μm, about 1 μm to about 8 μm, about 1 μm to about 5 μm, about 100 μm to about 1,500 μm, about 100 μm to about 1,200 μm, about 100 μm to about 1,000 μm, about 100 μm to about 800 μm, about 100 μm to about 600 μm, about 100 μm to about 500 μm, about 100 μm to about 400 μm, about 100 μm to about 300 μm, about 100 μm to about 200 μm, about 100 μm to about 150 μm, about 100 μm to about 120 μm, about 500 μm to about 1,500 μm, about 500 μm to about 1,200 μm, about 500 μm to about 1,000 μm, about 500 μm to about 800 μm, about 500 μm to about 600 μm, about 10 μm to about 1,000 μm, about 100 μm to about 1,000 μm, about 200 μm to about 1,000 μm, about 350 μm to about 1,000 μm, about 500 μm to about 1,000 μm, about 600 μm to about 1,000 μm, about 800 μm to about 1,000 μm, or about 900 μm to about 1,000 μm.

In some embodiments, the coating and/or the aluminum oxide coating has a thickness in a range from about 1 nm, about 2 nm, about 3 nm, about 4 nm, about 5 nm, about 6 nm, about 7 nm, about 8 nm, about 9 nm, about 10 nm, about 12 nm, about 15 nm, about 18 nm, or about 20 nm to about 22 nm, about 25 nm, about 30 nm, about 35 nm, about 40 nm, about 50 nm, about 60 nm, about 70 nm, about 80 nm, about 90 nm, about 95 nm, about 100 nm, or greater. For example, the coating and/or the aluminum oxide coating has a thickness in a range from about 1 nm to about 100 nm, about 2 nm to about 100 nm, about 3 nm to about 100 nm, about 5 nm to about 100 nm, about 6 nm to about 100 nm, about 8 nm to about 100 nm, about 10 nm to about 100 nm, about 12 nm to about 100 nm, about 15 nm to about 100 nm, about 18 nm to about 100 nm, about 20 nm to about 100 nm, about 30 nm to about 100 nm, about 40 nm to about 100 nm, about 50 nm to about 100 nm, about 60 nm to about 100 nm, or about 80 nm to about 100 nm.

In one or more embodiments, the coated particles and/or each coated particle can contain aluminum oxide in a range from about 0.2 wt %, about 0.3 wt %, about 0.5 wt %, about 0.8 wt %, about 1 wt %, about 1.2 wt %, about 1.5 wt %, about 1.8 wt %, about 2 wt %, about 2.5 wt %, about 3 wt %, about 3.5 wt %, or about 4 wt % to about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 15 wt %, about 20 wt %, or greater. For example, the coated particles and/or each coated particle can contain aluminum oxide in a range from about 0.5 wt % to about 12 wt %, about 0.5 wt % to about 10 wt %, about 0.5 wt % to about 8 wt %, about 0.5 wt % to about 7 wt %, about 0.5 wt % to about 6 wt %, about 0.5 wt % to about 5 wt %, about 0.5 wt % to about 4 wt %, about 0.5 wt % to about 3 wt %, about 0.5 wt % to about 2 wt %, about 0.5 wt % to about 1 wt %, about 1 wt % to about 12 wt %, about 1 wt % to about 10 wt %, about 1 wt % to about 8 wt %, about 1 wt % to about 7 wt %, about 1 wt % to about 6 wt %, about 1 wt % to about 5 wt %, about 1 wt % to about 4 wt %, about 1 wt % to about 3 wt %, about 1 wt % to about 2 wt %, about 1 wt % to about 1.5 wt %, about 3 wt % to about 12 wt %, about 3 wt % to about 10 wt %, about 3 wt % to about 8 wt %, about 3 wt % to about 7 wt %, about 3 wt % to about 6 wt %, about 3 wt % to about 5 wt %, or about 3 wt % to about 4 wt %.

In one or more embodiments, the coated particles and/or each coated particle can contain the protein powder particles in a range from about 30 wt %, about 40 wt %, about 50 wt %, about 60 wt %, about 70 wt %, about 80 wt %, about 85 wt %, about 88 wt %, about 90 wt %, about 91 wt %, about 92 wt %, about 93 wt %, about 94 wt %, or about 95 wt % to about 96 wt %, about 97 wt %, about 98 wt %, about 99 wt %, about 99.2 wt %, about 99.5 wt %, about 99.8 wt %, about 99.9 wt %, or about 99.95 wt %. For example, the coated particles and/or each coated particle can contain the protein powder particles in a range from about 30 wt % to about 99.8 wt %, about 50 wt % to about 99.8 wt %, about 70 wt % to about 99.8 wt %, about 85 wt % to about 99.8 wt %, about 88 wt % to about 99.8 wt %, about 90 wt % to about 99.8 wt %, about 92 wt % to about 99.8 wt %, about 95 wt % to about 99.8 wt %, about 96 wt % to about 99.8 wt %, about 90 wt % to about 99.5 wt %, about 92 wt % to about 99.5 wt %, about 93 wt % to about 99.5 wt %, about 95 wt % to about 99.5 wt %, about 96 wt % to about 99.5 wt %, about 97 wt % to about 99.5 wt %, about 98 wt % to about 99.5 wt %, about 90 wt % to about 99 wt %, about 92 wt % to about 99 wt %, about 93 wt % to about 99 wt %, about 95 wt % to about 99 wt %, about 96 wt % to about 99 wt %, about 97 wt % to about 99 wt %, about 98 wt % to about 99 wt %, about 90 wt % to about 95 wt %, about 92 wt % to about 95 wt %, or about 93 wt % to about 95 wt %.

In one or more examples, the coated particles and/or each coated particle can contain aluminum oxide in a range from about 0.2 wt % to about 15 wt % and the protein powder particles in a range from about 85 wt % to about 99.8 wt %. In other examples, the coated particles and/or each coated particle can contain aluminum oxide in a range from about 0.5 wt % to about 10 wt % and the protein powder particles in a range from about 90 wt % to about 99.5 wt %. In some examples, the coated particles and/or each coated particle can contain aluminum oxide in a range from about 1 wt % to about 7 wt % and the protein powder particles in a range from about 93 wt % to about 99 wt %.

In one or more embodiments, the plurality of coated particles has an average particle size in a range from about 0.1 μm, about 0.5 μm, about 1 μm, about 2 μm, about 5 μm, about 10 μm, about 20 μm, about 50 μm, about 80 μm, or about 100 μm to about 120 μm, about 150 μm, about 200 μm, about 250 μm, about 300 μm, about 400 μm, about 500 μm, about 600 μm, about 700 μm, about 800 μm, about 900 μm, about 950 μm, about 1,000 μm, or greater. For example, the plurality of coated particles has an average particle size in a range from about 0.1 μm to about 1,000 μm, about 1 μm to about 1,000 μm, about 1 μm to about 800 μm, about 1 μm to about 650 μm, about 1 μm to about 500 μm, about 1 μm to about 450 μm, about 1 μm to about 400 μm, about 1 μm to about 300 μm, about 1 μm to about 200 μm, about 1 μm to about 100 μm, about 1 μm to about 80 μm, about 1 μm to about 50 μm, about 1 μm to about 35 μm, about 1 μm to about 20 μm, about 1 μm to about 10 μm, about 1 μm to about 5 μm, about 10 μm to about 1,000 μm, about 10 μm to about 800 μm, about 10 μm to about 650 μm, about 10 μm to about 500 μm, about 10 μm to about 450 μm, about 10 μm to about 400 μm, about 10 μm to about 300 μm, about 10 μm to about 200 μm, about 10 μm to about 100 μm, about 10 μm to about 80 μm, about 10 μm to about 50 μm, about 10 μm to about 35 μm, about 10 μm to about 20 μm, about 100 μm to about 1,000 μm, about 100 μm to about 800 μm, about 100 μm to about 650 μm, about 100 μm to about 500 μm, about 100 μm to about 450 μm, about 100 μm to about 400 μm, about 100 μm to about 300 μm, about 100 μm to about 200 μm, or about 100 μm to about 150 μm.

### Atomic Layer Coating (ALC) Process

The ALC process described and discussed here for preparing the protein powder composition of coated particles which is produced by coating protein powder particles with a coating.

In one or more embodiments, methods for forming a protein powder composition are provided and include positioning a plurality of protein powder particles within a process region of a processing chamber, and coating the plurality of protein powder particles with an aluminum oxide coating to form a plurality of coated particles during an ALC process. The protein powder particles contain myoglobin. The ALC process includes one or more deposition cycles, and each of the deposition cycles includes exposing the plurality of protein powder particles to an aluminum precursor, infiltrating the plurality of protein powder particles with the aluminum precursor via spaces between the protein powder particles, purging the process region to remove gaseous remnants containing the aluminum precursor, exposing the plurality of protein powder particles to an oxidizing agent, infiltrating the plurality of protein powder particles with the oxidizing agent via spaces between the protein powder particles to produce the aluminum oxide coating disposed on outer surface of each of the protein powder particles, and purging the process region to remove gaseous remnants containing the oxidizing agent.

In one or more examples, the ALC process includes deposition cycles of exposing the protein powder particles to a precursor containing aluminum (e.g., one or more aluminum precursors), purging the process region to remove gaseous remnants containing the precursor, then exposing the protein powder particles to an oxidizing agent, to produce a coating containing aluminum oxide disposed on the surfaces of the protein powder particles, and then purging the process region to remove gaseous remnants containing the oxidizing agent.

Each of the ALC deposition cycles includes a first segment of exposing by a precursor, a second segment of purging the process region to remove remaining gaseous precursor, a third segment of exposing by an oxidizing agent, and a fourth segment of purging the process region to remove remaining gaseous precursor. One or more carrier gases can be flowed into the process region along with the precursor and/or the oxidizing agent during the first and third segments, respectively, of the ALC process. One or more purge gases can be flowed into the process region which is also being evacuated during the second and fourth segments of the ALC process. The carrier gas and the purge gas can be the same composition or different compositions. Exemplary carrier gases and/or purge gases can be or include argon, helium, neon, nitrogen (N₂), hydrogen (H₂), or any combination thereof.

The process region of the processing chamber is the internal volume within the processing chamber. The process region and/or the internal volume of the processing chamber are maintained at and/or adjusted to one or more pressures below atmospheric or ambient pressure (e.g., less than 760 Torr) during the ALC process. The pressure of the process region and/or the internal volume of the processing chamber is at about 0.01 Torr, about 0.1 Torr, about 1 Torr, about 1 Torr, about 5 Torr, about 10 Torr, about 15 Torr, about 20 Torr, about 25 Torr, about 35 Torr, or about 50 Torr to about 80 Torr, about 100 Torr, about 150 Torr, about 200 Torr, about 250 Torr, about 300 Torr, about 350 Torr, about 400 Torr, about 450 Torr, about 500 Torr, or about 600 Torr during the ALC process.

In one or more embodiments, the process region and/or the internal volume of the processing chamber are maintained at and/or adjusted to a pressure in a range from about 0.1 Torr, about 0.5 Torr, about 0.8 Torr, or about 1 Torr to about 1.5 Torr, about 3 Torr, about 5 Torr, about 8 Torr, about 10 Torr, about 15 Torr, or greater during the ALC process. For example, process region and/or the internal volume of the processing chamber are maintained at and/or adjusted to a pressure in a range from about 0.1 Torr to about 15 Torr, about 0.1 Torr to about 10 Torr, about 0.1 Torr to about 5 Torr, about 0.1 Torr to about 1 Torr, about 1 Torr to about 15 Torr, about 1 Torr to about 10 Torr, or about 1 Torr to about 5 Torr during the ALC process.

The process region, the internal volume of the processing chamber, and/or the protein powder particles are maintained at and/or adjusted to one or more process temperature during the ALC process. The process temperature can be in a range from about 20° C., about 23° C., about 25° C., about 30° C., about 35° C., or about 40° C. to about 50° C., about 60° C., about 70° C., about 75° C., about 80° C., about 90° C., about 100° C., or greater during the ALC process. For example, the process temperature can be in a range from about 20° C. to about 100° C., about 30° C. to about 100° C., about 50° C. to about 100° C., about 65° C. to about 100° C., about 70° C. to about 100° C., about 75° C. to about 100° C., about 80° C. to about 100° C., about 20° C. to about 80° C., about 30° C. to about 80° C., about 50° C. to about 80° C., about 65° C. to about 80° C., about 70° C. to about 80° C., about 75° C. to about 80° C., about 80° C. to about 85° C., about 20° C. to about 60° C., about 25° C. to about 60° C., about 30° C. to about 60° C., about 35° C. to about 60° C., about 40° C. to about 60° C., about 45° C. to about 60° C., or about 50° C. to about 60° C. during the ALC process.

Each of the first segment and the third segment of the ALC deposition cycle can independently last about 20 seconds, about 30 seconds, about 35 seconds, about 40 seconds, or about 45 seconds to about 50 seconds, about 60 seconds, about 70 seconds, about 80 seconds, about 90 seconds, about 100 seconds, about 2 minutes, about 2.5 minutes, about 3 minutes, about 4 minutes, about 5 minutes, about 6 minutes, about 8 minutes, about 10 minutes, about 12 minutes, about 15 minutes, about 18 minutes, about 20 minutes, about 25 minutes, or about 30 minutes during the ALC process. For example, each of the first segment and the third segment of the ALC deposition cycle can independently last about 20 seconds to about 30 minutes, about 20 seconds to about 25 minutes, about 20 seconds to about 20 minutes, about 20 seconds to about 15 minutes, about 20 seconds to about 12 minutes, about 20 seconds to about 10 minutes, about 20 seconds to about 8 minutes, about 20 seconds to about 6 minutes, about 20 seconds to about 5 minutes, about 20 seconds to about 4 minutes, about 20 seconds to about 3 minutes, about 20 seconds to about 2.5 minutes, about 20 seconds to about 2 minutes, about 20 seconds to about 100 seconds, about 20 seconds to about 90 seconds, about 20 seconds to about 75 seconds, about 20 seconds to about 60 seconds, about 20 seconds to about 45 seconds, about 20 seconds to about 30 seconds, about 40 seconds to about 5 minutes, about 40 seconds to about 4 minutes, about 40 seconds to about 3 minutes, about 40 seconds to about 2.5 minutes, about 40 seconds to about 2 minutes, about 40 seconds to about 100 seconds, about 40 seconds to about 90 seconds, about 40 seconds to about 75 seconds, about 40 seconds to about 60 seconds, about 60 seconds to about 20 minutes, about 60 seconds to about 15 minutes, about 60 seconds to about 12 minutes, about 60 seconds to about 10 minutes, about 60 seconds to about 8 minutes, about 60 seconds to about 6 minutes, about 60 seconds to about 5 minutes, about 60 seconds to about 4 minutes, about 60 seconds to about 3 minutes, about 60 seconds to about 2.5 minutes, about 60 seconds to about 2 minutes, about 60 seconds to about 100 seconds, about 60 seconds to about 90 seconds, or about 60 seconds to about 75 seconds during the ALC process.

In one or more examples, the protein powder particles are exposed to the aluminum precursor for about 0.1 minute to about 30 minutes during the first segment of each ALC deposition cycle. In some examples, the protein powder particles are exposed to the oxidizing agent for about 1 minute to about 30 minutes during the third segment of each ALC deposition cycle.

Each of the second segment and the fourth segment of the ALC deposition cycle can independently last about 20 seconds, about 30 seconds, about 35 seconds, about 40 seconds, about 45 seconds, about 50 seconds, about 60 seconds, about 70 seconds, about 80 seconds, or about 90 seconds, about 100 seconds, about 2 minutes, about 2.5 minutes, about 3 minutes, about 4 minutes, about 5 minutes, about 6 minutes, about 8 minutes, about 10 minutes, about 12 minutes, about 15 minutes, about 20 minutes, about 25 minutes, or about 30 minutes during the ALC process. For example, each of the second segment and the fourth segment of the ALC deposition cycle can independently last about 20 seconds to about 30 minutes, about 20 seconds to about 25 minutes, about 20 seconds to about 20 minutes, about 20 seconds to about 15 minutes, about 20 seconds to about 12 minutes, about 20 seconds to about 10 minutes, about 20 seconds to about 8 minutes, about 20 seconds to about 6 minutes, about 20 seconds to about 5 minutes, about 20 seconds to about 4 minutes, about 20 seconds to about 3 minutes, about 20 seconds to about 2.5 minutes, about 20 seconds to about 2 minutes, about 20 seconds to about 100 seconds, about 20 seconds to about 90 seconds, about 20 seconds to about 75 seconds, about 20 seconds to about 60 seconds, about 20 seconds to about 45 seconds, about 20 seconds to about 30 seconds, about 40 seconds to about 5 minutes, about 40 seconds to about 4 minutes, about 40 seconds to about 3 minutes, about 40 seconds to about 2.5 minutes, about 40 seconds to about 2 minutes, about 40 seconds to about 100 seconds, about 40 seconds to about 90 seconds, about 40 seconds to about 75 seconds, about 40 seconds to about 60 seconds, about 60 seconds to about 20 minutes, about 60 seconds to about 15 minutes, about 60 seconds to about 12 minutes, about 60 seconds to about 10 minutes, about 60 seconds to about 8 minutes, about 60 seconds to about 6 minutes, about 60 seconds to about 5 minutes, about 60 seconds to about 4 minutes, about 60 seconds to about 3 minutes, about 60 seconds to about 2.5 minutes, about 60 seconds to about 2 minutes, about 60 seconds to about 100 seconds, about 60 seconds to about 90 seconds, or about 60 seconds to about 75 seconds during the ALC process.

In one or more examples, the protein powder particles are exposed to a purge gas for about 1 minute to about 30 minutes while purging the process region to remove the gaseous remnants containing the aluminum precursor and/or any byproducts during the second segment of each ALC deposition cycle. In other examples, the protein powder particles are exposed to a purge gas for about 1 minute to about 30 minutes while purging the process region to remove the gaseous remnants containing the oxidizing agent and/or any byproducts during the fourth segment of each ALC deposition cycle.

In some examples, the plurality of protein powder particles is exposed to the aluminum precursor for about 0.1 minutes to about 30 minutes while infiltrating the plurality of protein powder particles with the aluminum precursor during each of the deposition cycles. Thereafter, the plurality of protein powder particles is exposed to a purge gas for about 1 minute to about 30 minutes while purging the process region to remove the gaseous remnants containing the aluminum precursor during each of the deposition cycles. Thereafter, the plurality of protein powder particles is exposed to the oxidizing agent for about 1 minute to about 10 minutes while infiltrating the plurality of protein powder particles with the aluminum precursor during each of the deposition cycles. Thereafter, the plurality of protein powder particles is exposed to a purge gas for about 1 minute to about 30 minutes while purging the process region to remove the gaseous remnants containing the oxidizing agent during each of the deposition cycles.

The ALC deposition cycle can be conducted once or multiple times while coating the protein powder particles. In one or more embodiments, the ALC deposition cycle is repeated in a range from 1 time, 2 times, 3 times, 5 times, 8 times, 10 times, about 15 times, about 20 times, about 30 times, or about 40 times to about 45 times, about 50 times, about 60 times, about 70 times, about 80 times, about 90 times, about 100 times, about 120 times, about 150 times, about 180 times, about 200 times, or more during the ALC process. For example, the ALC deposition cycle can be repeated in a range from 1 time to about 200 times, 1 time to about 150 times, 1 time to about 100 times, 1 time to about 80 times, 1 time to about 50 times, 1 time to about 20 times, 1 time to about 10 times, 1 time to 5 times, 5 times to about 200 times, 5 times to about 150 times, 5 times to about 100 times, 5 times to about 80 times, 5 times to about 50 times, 5 times to about 20 times, 5 times to about 10 times, 10 times to about 200 times, 10 times to about 150 times, 10 times to about 100 times, 10 times to about 80 times, 10 times to about 50 times, or 10 times to about 20 times during the ALC process.

The precursor exposed to the protein powder particles can be one or more aluminum precursors or other metal precursors. The oxidizing agent can be or include any compound or reagent which will oxidize the aluminum precursor to produce aluminum oxide or another metal precursor to produce the respective metal oxide. The oxidizing agent can be or include water (including water vapor or steam), ozone, oxygen plasma, oxygen radicals, oxygen (O₂), hydrogen peroxide, or any combination thereof.

In one or more embodiments, the precursor is or includes one or more aluminum precursors used to coat the protein powder particles with a coating containing aluminum oxide. The aluminum precursor can be or include one or more alkylaluminum compounds, one or more alkoxyaluminum compounds, one or more aluminum halide compounds, aluminum hydrides, or any combination thereof. In some examples, the aluminum precursor is or contains trimethyl aluminum, triethyl aluminum, tripropyl aluminum, tributyl aluminum, dimethyl aluminum, diethyl aluminum, dipropyl aluminum, dibutyl aluminum, complexes thereof, or combinations thereof.

In one or more examples, the aluminum precursor is or contains one or more alkylaluminum compounds (e.g., trimethylaluminum) and the oxidizing agent is or includes water. In other examples, the aluminum precursor is or contains one or more alkoxyaluminum compounds and the oxidizing agent is or contains ozone or oxygen plasma.

### Experimental

Experiments were conducted and properties were measured for various samples of protein powders. A soluble protein recovery procedure followed the Size-Exclusion Chromatography (SEC) procedure as follows:

Between 2-3 mg of the solid protein formulation was weighed out and reconstituted in H₂O at a concentration of 2 mg/mL. This was then centrifuged to remove insoluble material at 13,100 g for 10 minutes. The supernatant (0.75 mL) was transferred to a 1.5 mL HPLC vial with septum cap. SEC measurements were run using a PerkinElmer LC300 HPLC equipped with a PDA detector. Protein visible absorption band at 410 nm was used to detect the protein. The solution (30 μL) was injected onto an TSKgel® 3000 size-exclusion column (Tosoh Bioscience). The mobile phase was made up of phosphate buffer (50 mM) with NaCl (100 mM) and pH adjusted to 7.1. Each run was carried out using isocratic elution at a flow rate of 0.5 mL/min and a total run time of 30 minutes. Three repeat injections were carried out for each sample. Concentrations of monomeric myoglobin and its soluble aggregates were determined using a 5-point calibration curve prepared using a myoglobin standard. Molar absorptivities of soluble myoglobin aggregates were assumed equal to that of monomeric myoglobin.

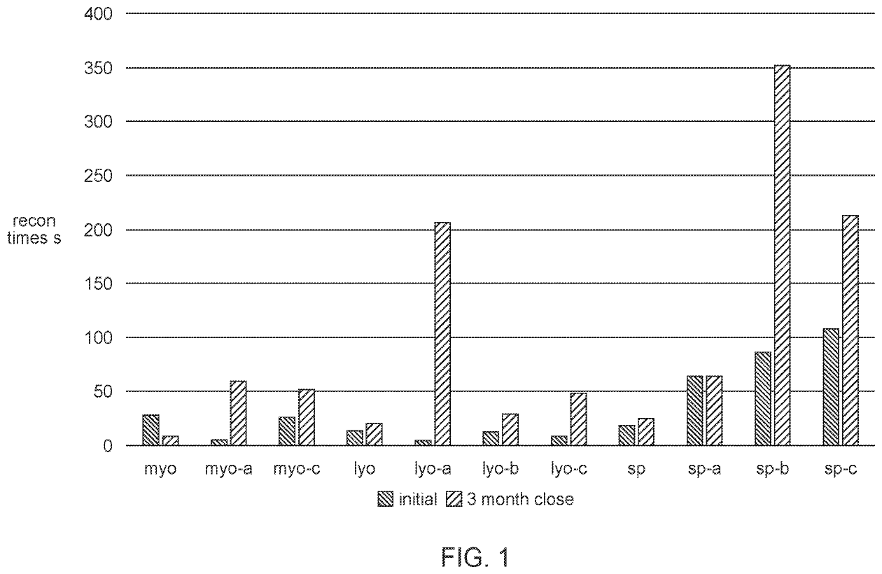

In FIGS. 1-8, the samples of protein powders are labeled as follows: “myo” is crude myoglobin powder, “lyo” is lyophilized myoglobin, and “sp” is spray dried myoglobin. The protein powder sample was coated if the label is followed by a hyphenated “a”, “b”, or “c”—such as myo-a, myo-b, myo-c, lyo-a, lyo-b, lyo-c, sp-a, sp-b, and sp-c are coated samples. The coating thickness is indicative to “a” is less than “b” is less than “c”. The protein powder sample was uncoated if the was no hyphenated “a”, “b”, or “c”—such as myo, lyo, and sp are uncoated samples.

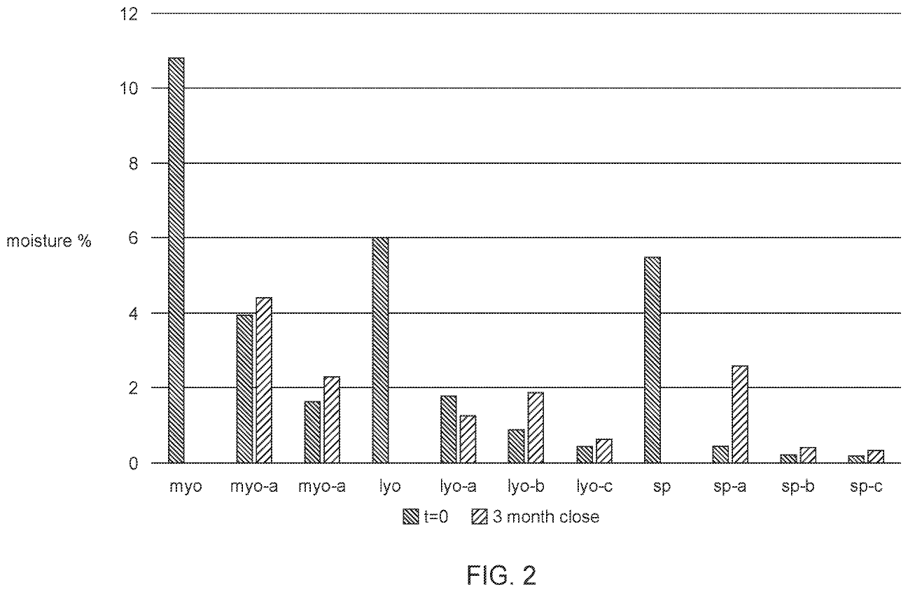

FIG. 1 is a graph illustrating reconstitution time for a variety of samples of protein powders and FIG. 2 is a graph illustrating moisture concentration for a variety of samples of protein powders. There was no clear trend of moisture content and recon time between the samples of protein powders. The recon time after accelerating test increased but still within acceptable ranges.

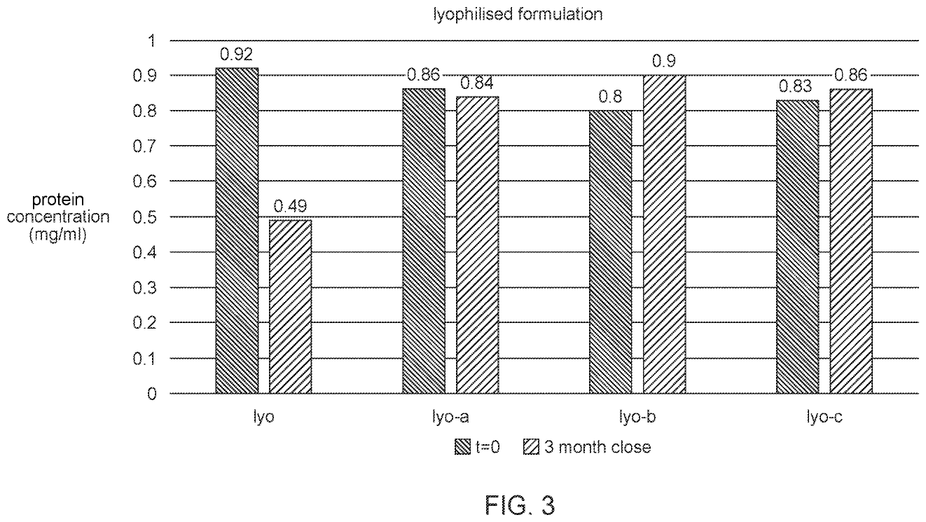

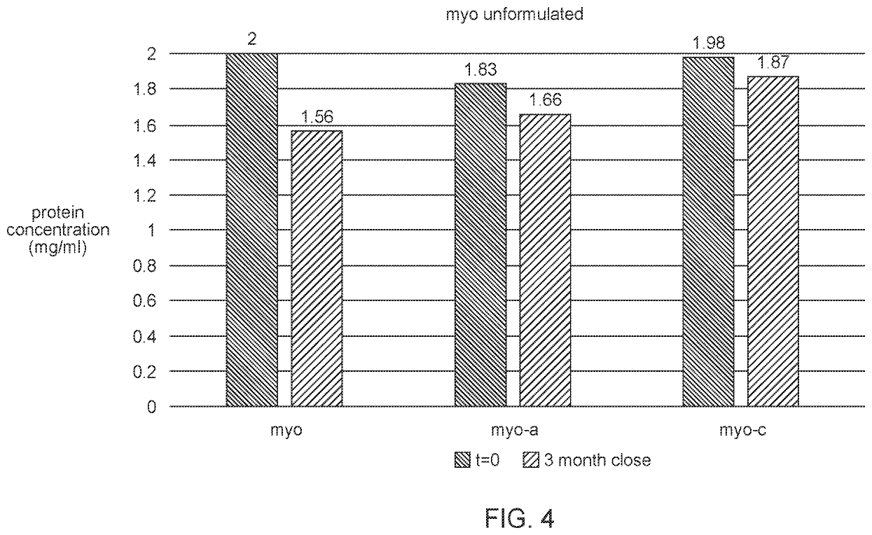

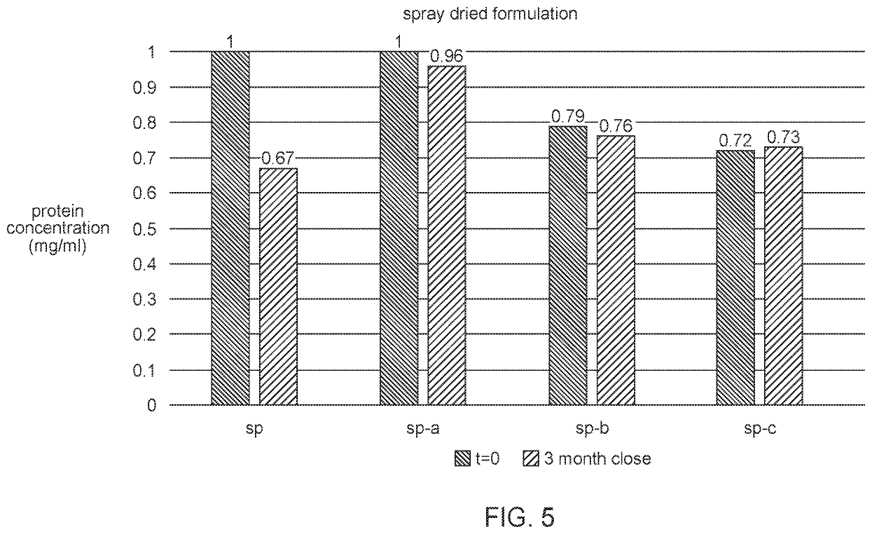

FIG. 3 is a graph illustrating soluble protein recovery for samples of lyophilized protein powders, FIG. 4 is a graph illustrating soluble protein recovery for samples of unformulated protein powders, and FIG. 5 is a graph illustrating soluble protein recovery for samples of spray dried protein powders.

For both coated lyo, sp samples showed very good protein recovery after the 3-month accelerating test. Uncoated lyo, sp samples showed 30%-50% protein loss. Unformulated myoglobin samples, coated samples showed slightly better recovery than uncoated samples.

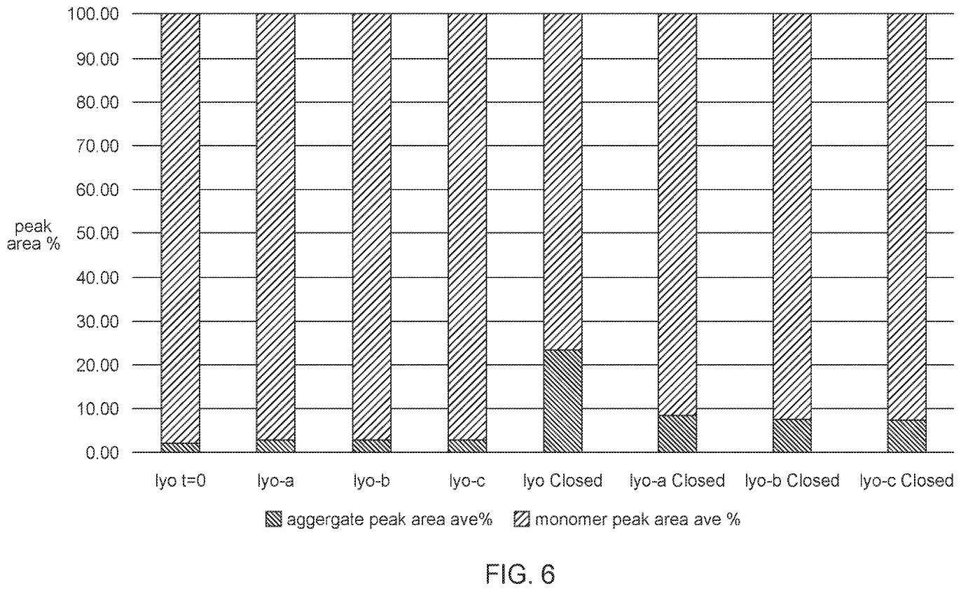

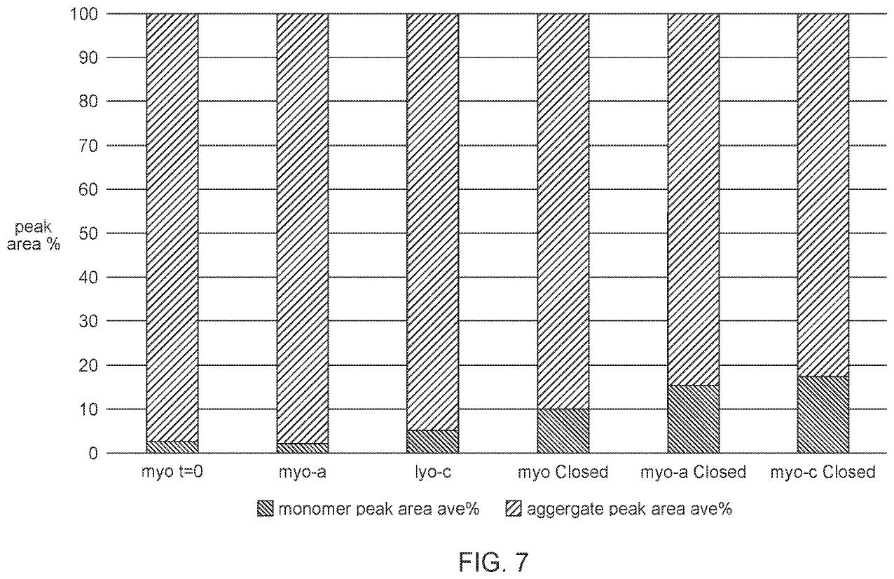

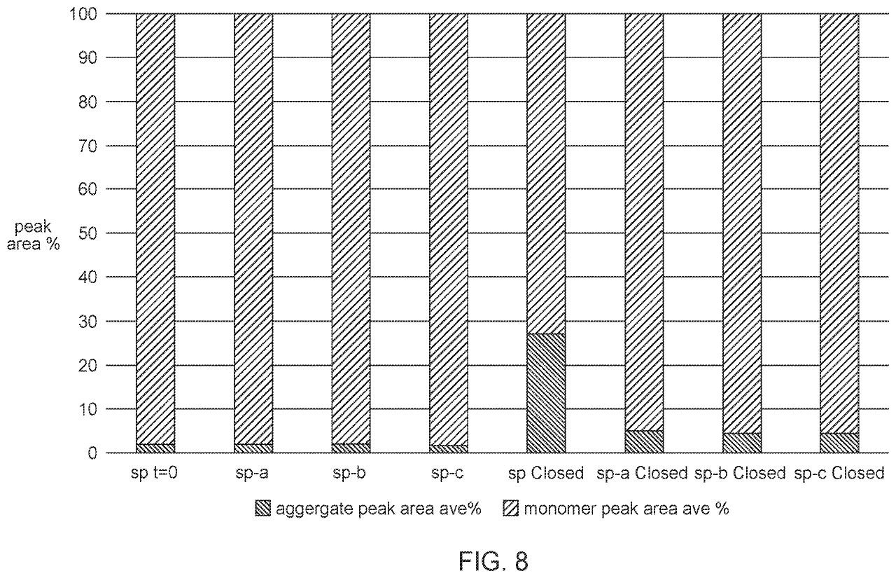

FIG. 6 is a graph illustrating the relative quantity of aggregates in soluble protein for samples of lyophilized protein powders, FIG. 7 is a graph illustrating the relative quantity of aggregates in soluble protein for samples of unformulated protein powders, and FIG. 8 is a graph illustrating the relative quantity of aggregates in soluble protein for samples of spray dried protein powders.

At to, coated and uncoated lyo, sp samples had similar aggregation %, about 2%-3%. Both coated sp and lyo samples showed much less % of aggregation. Less than 5% aggregation was determined for coated sp samples. About 27% aggregation was determined for uncoated sp samples. Less than 10% aggregation was determined for the coated lyo sample. About 23% aggregation was determined for uncoated lyo samples.

Most traditional chemical vapor deposition (CVD) chambers or atomic layer deposition (ALD) chambers can be used as the processing chamber suitable for performing an atomic layer coating (ALC) process described and discussed herein. One example of the processing chamber that may be adapted to benefit from the ALC process is a CENTRIS® Sym3™ etching processing chamber, commercially available from Applied Materials, Inc. An example of a tool or system that benefit from the ALC process is the Centura® system or Endura® system with an iSprint™ ALD/CVD SSW chamber, commercially available from Applied Materials, Inc.

Embodiments of the present disclosure further relate to any one or more of the following Embodiments 1-67:
1. A method of forming a protein powder composition, comprising: positioning a plurality of protein powder particles within a process region of a processing chamber, wherein each of the protein powder particles comprises myoglobin; and coating the plurality of protein powder particles with an aluminum oxide coating to form a plurality of coated particles during an atomic layer coating (ALC) process, wherein the ALC process comprises one or more deposition cycles, and each of the deposition cycles comprises: exposing the plurality of protein powder particles to an aluminum precursor; infiltrating the plurality of protein powder particles with the aluminum precursor via spaces between the protein powder particles; purging the process region to remove gaseous remnants containing the aluminum precursor; exposing the plurality of protein powder particles to an oxidizing agent; infiltrating the plurality of protein powder particles with the oxidizing agent via spaces between the protein powder particles to produce the aluminum oxide coating disposed on outer surface of each of the protein powder particles; and purging the process region to remove gaseous remnants containing the oxidizing agent.
2. The method according to Embodiment 1, wherein the plurality of coated particles has a greater flowability value than the plurality of protein powder particles.
3. The method according to Embodiment 1 or 2, wherein the plurality of coated particles has a greater bulk density than the plurality of protein powder particles.
4. The method according to any one of Embodiments 1-3, wherein each of the protein powder particles further comprises a sugar.
5. The method according to Embodiment 4, wherein the sugar comprises sucrose, mannitol, lactose, trehalose, sorbitol, isomers thereof, derivatives thereof, salts thereof, or any combination thereof.
6. The method according to Embodiment 4, wherein each of the coated particles comprises about 0.01 wt % to about 15 wt % of the sugar.
7. The method according to any one of Embodiments 1-6, wherein each of the protein powder particles further comprises a buffer.
8. The method according to Embodiment 8, wherein the buffer comprises potassium phosphate.
9. The method according to any one of Embodiments 1-8, wherein each of the protein powder particles further comprises an additive and/or a component selected from one or more surfactants, one or more isotonicity agents, one or more antioxidants, one or more chelators, one or more preservatives, one or more amino acids, one or more monomers, one or more polymers, derivatives thereof, or any combination thereof.
10. The method according to any one of Embodiments 1-9, wherein the plurality of protein powder particles has an average particle size of about 0.1 μm to about 1,000 μm.
11. The method according to any one of Embodiments 1-10, wherein the plurality of protein powder particles are produced from a spray dried process or a lyophilization process.
12. The method according to any one of Embodiments 1-11, wherein the aluminum oxide coating has a thickness of about 1 nm to about 100 nm.
13. The method according to any one of Embodiments 1-12, wherein each of the coated particles comprises about 0.5 wt % to about 10 wt % of aluminum oxide.
14. The method according to any one of Embodiments 1-13, wherein each of the coated particles comprises about 1 wt % to about 7 wt % of aluminum oxide.
15. The method according to any one of Embodiments 1-14, wherein each of the coated particles comprises about 90 wt % to about 99.5 wt % of the protein powder particles comprising myoglobin.
16. The method according to any one of Embodiments 1-15, wherein each of the coated particles comprises about 93 wt % to about 99 wt % of the protein powder particles comprising myoglobin.
17. The method according to any one of Embodiments 1-16, wherein the plurality of coated particles has an average particle size of about 0.1 μm to about 1,000 μm.
18. The method according to any one of Embodiments 1-17, wherein the aluminum precursor comprises an alkylaluminum compound.
19. The method according to any one of Embodiments 1-18, wherein the oxidizing agent comprises water, oxygen (O₂), ozone, atomic oxygen, oxygen plasma, hydrogen peroxide, or any combination thereof.
20. The method according to any one of Embodiments 1-19, wherein the aluminum precursor comprises trimethyl aluminum and the oxidizing agent comprises water.
21. The method according to any one of Embodiments 1-20, wherein the deposition cycle is repeated 1 time to about 100 times during the ALC process.
22. The method according to any one of Embodiments 1-21, wherein the process region of the processing chamber is at a pressure of about 0.5 Torr to about 10 Torr during the ALC process.
23. The method according to any one of Embodiments 1-22, wherein the plurality of protein powder particles is exposed to the aluminum precursor for about 0.1 minutes to about 30 minutes while infiltrating the plurality of protein powder particles with the aluminum precursor during each of the deposition cycles.
24. The method according to any one of Embodiments 1-23, wherein the plurality of protein powder particles is exposed to a purge gas for about 1 minute to about 30 minutes while purging the process region to remove the gaseous remnants containing the aluminum precursor during each of the deposition cycles.
25. The method according to any one of Embodiments 1-24, wherein the plurality of protein powder particles is exposed to the oxidizing agent for about 1 minute to about 10 minutes while infiltrating the plurality of protein powder particles with the aluminum precursor during each of the deposition cycles.
26. The method according to any one of Embodiments 1-25, wherein the plurality of protein powder particles is exposed to a purge gas for about 1 minute to about 30 minutes while purging the process region to remove the gaseous remnants containing the oxidizing agent during each of the deposition cycles.
27. A method of forming a protein powder composition, comprising: positioning a plurality of protein powder particles within a process region of a processing chamber, wherein each of the protein powder particles comprises myoglobin; and coating the plurality of protein powder particles with an aluminum oxide coating to form a plurality of coated particles during an atomic layer coating (ALC) process, wherein the plurality of coated particles has a greater flowability value than the plurality of protein powder particles, and wherein the ALC process comprises one or more deposition cycles, and each of the deposition cycles comprises: exposing the plurality of protein powder particles to an aluminum precursor; purging the process region to remove gaseous remnants containing the aluminum precursor; exposing the plurality of protein powder particles to an oxidizing agent; and purging the process region to remove gaseous remnants containing the oxidizing agent.
28. The method according to Embodiment 27, wherein the plurality of coated particles has a greater bulk density than the plurality of protein powder particles.
29. The method according to Embodiment 27 or 28, wherein each of the protein powder particles further comprises a sugar.
30. The method according to Embodiment 29, wherein the sugar comprises sucrose, mannitol, lactose, trehalose, sorbitol, isomers thereof, derivatives thereof, salts thereof, or any combination thereof.
31. The method according to Embodiment 29, wherein each of the coated particles comprises about 0.01 wt % to about 15 wt % of the sugar.
32. The method according to any one of Embodiments 27-31, wherein each of the protein powder particles further comprises a buffer.
33. The method according to Embodiment 32, wherein the buffer comprises potassium phosphate.
34. The method according to any one of Embodiments 27-33, wherein each of the protein powder particles further comprises an additive and/or a component selected from one or more surfactants, one or more isotonicity agents, one or more antioxidants, one or more chelators, one or more preservatives, one or more amino acids, one or more monomers, one or more polymers, derivatives thereof, or any combination thereof.
35. The method according to any one of Embodiments 27-34, wherein the plurality of protein powder particles has an average particle size of about 0.1 μm to about 1,000 μm.
36. The method according to any one of Embodiments 27-35, wherein the plurality of protein powder particles are produced from a spray dried process or a lyophilization process.
37. The method according to any one of Embodiments 27-36, wherein the aluminum oxide coating has a thickness of about 1 nm to about 100 nm.
38. The method according to any one of Embodiments 27-37, wherein each of the coated particles comprises about 0.5 wt % to about 10 wt % of aluminum oxide.
39. The method according to any one of Embodiments 27-38, wherein each of the coated particles comprises about 1 wt % to about 7 wt % of aluminum oxide.
40. The method according to any one of Embodiments 27-39, wherein each of the coated particles comprises about 90 wt % to about 99.5 wt % of the protein powder particles comprising myoglobin.
41. The method according to any one of Embodiments 27-40, wherein each of the coated particles comprises about 93 wt % to about 99 wt % of the protein powder particles comprising myoglobin.
42. The method according to any one of Embodiments 27-41, wherein the plurality of coated particles has an average particle size of about 0.1 μm to about 1,000 μm.
43. The method according to any one of Embodiments 27-42, wherein the aluminum precursor comprises an alkylaluminum compound.
44. The method according to any one of Embodiments 27-43, wherein the oxidizing agent comprises water, oxygen (O₂), ozone, atomic oxygen, oxygen plasma, hydrogen peroxide, or any combination thereof.
45. The method according to any one of Embodiments 27-44, wherein the aluminum precursor comprises trimethyl aluminum and the oxidizing agent comprises water.
46. The method according to any one of Embodiments 27-45, wherein the deposition cycle is repeated 1 time to about 100 times during the ALC process.
47. The method according to any one of Embodiments 27-46, wherein the process region of the processing chamber is at a pressure of about 0.5 Torr to about 10 Torr during the ALC process.
48. The method according to any one of Embodiments 27-47, wherein the plurality of protein powder particles is exposed to the aluminum precursor for about 0.1 minutes to about 30 minutes while infiltrating the plurality of protein powder particles with the aluminum precursor during each of the deposition cycles.
49. The method according to any one of Embodiments 27-48, wherein the plurality of protein powder particles is exposed to a purge gas for about 1 minute to about 30 minutes while purging the process region to remove the gaseous remnants containing the aluminum precursor during each of the deposition cycles.
50. The method according to any one of Embodiments 27-49, wherein the plurality of protein powder particles is exposed to the oxidizing agent for about 1 minute to about 10 minutes while infiltrating the plurality of protein powder particles with the aluminum precursor during each of the deposition cycles.
51. The method according to any one of Embodiments 27-50, wherein the plurality of protein powder particles is exposed to a purge gas for about 1 minute to about 30 minutes while purging the process region to remove the gaseous remnants containing the oxidizing agent during each of the deposition cycles.
52. A protein powder composition, comprising: a plurality of coated particles, wherein each coated particles comprises: a protein powder particle comprising myoglobin; and a coating disposed around each of the protein powder particles, wherein the coating comprises aluminum oxide.
53. The protein powder composition according to Embodiment 52, wherein the coating is formed on the protein powder particles during an atomic layer coating (ALC) process.
54. The protein powder composition according to Embodiment 52 or 53, wherein each of the protein powder particles further comprises a sugar.
55. The protein powder composition according to Embodiment 54, wherein the sugar comprises sucrose, mannitol, lactose, trehalose, sorbitol, isomers thereof, derivatives thereof, salts thereof, or any combination thereof.
56. The protein powder composition according to Embodiment 54, wherein each of the coated particles comprises about 0.01 wt % to about 15 wt % of the sugar.
57. The protein powder composition according to any one of Embodiments 52-56, wherein each of the protein powder particles further comprises a buffer.
58. The protein powder composition according to Embodiment 57, wherein the buffer comprises potassium phosphate.
59. The protein powder composition according to any one of Embodiments 52-58, wherein each of the protein powder particles further comprises an additive and/or a component selected from one or more surfactants, one or more isotonicity agents, one or more antioxidants, one or more chelators, one or more preservatives, one or more amino acids, one or more monomers, one or more polymers, derivatives thereof, or any combination thereof.
60. The protein powder composition according to any one of Embodiments 52-59, wherein the plurality of protein powder particles has an average particle size of about 0.1 μm to about 1,000 μm.
61. The protein powder composition according to any one of Embodiments 52-60, wherein the plurality of protein powder particles are produced from a spray dried process or a lyophilization process.
62. The protein powder composition according to any one of Embodiments 52-61, wherein the aluminum oxide coating has a thickness of about 1 nm to about 100 nm.
63. The protein powder composition according to any one of Embodiments 52-62, wherein each of the coated particles comprises about 0.5 wt % to about 10 wt % of aluminum oxide.
64. The protein powder composition according to any one of Embodiments 52-63, wherein each of the coated particles comprises about 1 wt % to about 7 wt % of aluminum oxide.
65. The protein powder composition according to any one of Embodiments 52-64, wherein each of the coated particles comprises about 90 wt % to about 99.5 wt % of the protein powder particles comprising myoglobin.
66. The protein powder composition according to any one of Embodiments 52-65, wherein each of the coated particles comprises about 93 wt % to about 99 wt % of the protein powder particles comprising myoglobin.
67. The protein powder composition according to any one of Embodiments 52-66, wherein the plurality of coated particles has an average particle size of about 0.1 μm to about 1,000 μm.

While the foregoing is directed to embodiments of the disclosure, other and further embodiments may be devised without departing from the basic scope thereof, and the scope thereof is determined by the claims that follow. All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise, whenever a composition, an element, or a group of elements is preceded with the transitional phrase “comprising”, it is understood that the same composition or group of elements with transitional phrases “consisting essentially of”, “consisting of”, “selected from the group of consisting of”, or “is” preceding the recitation of the composition, element, or elements and vice versa, are contemplated. As used herein, the term “about” refers to a +/−10% variation from the nominal value. It is to be understood that such a variation can be included in any value provided herein.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below.

What is claimed is:

1. A method of forming a protein powder composition, comprising:
positioning a plurality of protein powder particles within a process region of a processing chamber, wherein each of the protein powder particles comprises myoglobin; and
coating the plurality of protein powder particles with an aluminum oxide coating to form a plurality of coated particles during an atomic layer coating (ALC) process, wherein the ALC process comprises one or more deposition cycles, and each of the deposition cycles comprises:
exposing the plurality of protein powder particles to an aluminum precursor;
infiltrating the plurality of protein powder particles with the aluminum precursor via spaces between the protein powder particles;
purging the process region to remove gaseous remnants containing the aluminum precursor;
exposing the plurality of protein powder particles to an oxidizing agent;
infiltrating the plurality of protein powder particles with the oxidizing agent via spaces between the protein powder particles to produce the aluminum oxide coating disposed on outer surface of each of the protein powder particles; and
purging the process region to remove gaseous remnants containing the oxidizing agent.

2. The method of claim 1, wherein the plurality of coated particles has a greater bulk density than the plurality of protein powder particles.

3. The method of claim 1, wherein each of the protein powder particles further comprises a sugar, and wherein the sugar comprises sucrose, mannitol, lactose, trehalose, sorbitol, isomers thereof, derivatives thereof, salts thereof, or any combination thereof.

4. The method of claim 3, wherein each of the coated particles comprises about 0.01 wt % to about 15 wt % of the sugar.

5. The method of claim 1, wherein each of the protein powder particles further comprises a buffer, and wherein the buffer comprises potassium phosphate.

6. The method of claim 1, wherein each of the protein powder particles further comprises an additive and/or a component selected from one or more surfactants, one or more isotonicity agents, one or more antioxidants, one or more chelators, one or more preservatives, one or more amino acids, one or more monomers, one or more polymers, derivatives thereof, or any combination thereof.

7. The method of claim 1, wherein the plurality of protein powder particles has an average particle size of about 0.1 μm to about 1,000 μm.

8. The method of claim 1, wherein the plurality of protein powder particles are produced from a spray dried process or a lyophilization process.

9. The method of claim 1, wherein the aluminum oxide coating has a thickness of about 1 nm to about 100 nm.

10. The method of claim 1, wherein each of the coated particles comprises about 0.5 wt % to about 10 wt % of aluminum oxide, and wherein each of the coated particles comprises about 90 wt % to about 99.5 wt % of the protein powder particles comprising myoglobin.

11. The method of claim 1, wherein the plurality of coated particles has an average particle size of about 0.1 μm to about 1,000 μm.

12. The method of claim 1, wherein the aluminum precursor comprises trimethyl aluminum and the oxidizing agent comprises water.

13. The method of claim 1, wherein the deposition cycle is repeated 1 time to about 100 times during the ALC process.

14. The method of claim 1, wherein:
the plurality of protein powder particles is exposed to the aluminum precursor for about 0.1 minutes to about 30 minutes while infiltrating the plurality of protein powder particles with the aluminum precursor during each of the deposition cycles;
the plurality of protein powder particles is exposed to a purge gas for about 1 minute to about 30 minutes while purging the process region to remove the gaseous remnants containing the aluminum precursor during each of the deposition cycles;
the plurality of protein powder particles is exposed to the oxidizing agent for about 1 minute to about 10 minutes while infiltrating the plurality of protein powder particles with the aluminum precursor during each of the deposition cycles; and
the plurality of protein powder particles is exposed to a purge gas for about 1 minute to about 30 minutes while purging the process region to remove the gaseous remnants containing the oxidizing agent during each of the deposition cycles.

15. A method of forming a protein powder composition, comprising:
positioning a plurality of protein powder particles within a process region of a processing chamber, wherein each of the protein powder particles comprises myoglobin; and
coating the plurality of protein powder particles with an aluminum oxide coating to form a plurality of coated particles during an atomic layer coating (ALC) process, wherein the plurality of coated particles has a greater flowability value than the plurality of protein powder particles, and wherein the ALC process comprises one or more deposition cycles, and each of the deposition cycles comprises:
exposing the plurality of protein powder particles to an aluminum precursor;
purging the process region to remove gaseous remnants containing the aluminum precursor;
exposing the plurality of protein powder particles to an oxidizing agent; and
purging the process region to remove gaseous remnants containing the oxidizing agent.

16. The method of claim 15, wherein the plurality of protein powder particles are produced from a spray dried process or a lyophilization process.

17. The method of claim 15, wherein each of the coated particles comprises about 0.5 wt % to about 10 wt % of aluminum oxide, and wherein each of the coated particles comprises about 90 wt % to about 99.5 wt % of the protein powder particles comprising myoglobin.

18. The method of claim 15, wherein the aluminum precursor comprises trimethyl aluminum and the oxidizing agent comprises water, wherein the deposition cycle is repeated 1 time to about 100 times during the ALC process.

19. A protein powder composition, comprising:
a plurality of coated particles, wherein each coated particles comprises:
a protein powder particle comprising myoglobin; and
a coating disposed around each of the protein powder particles, wherein the coating comprises aluminum oxide.

20. The protein powder composition of claim 19, wherein:
the coating is formed on the protein powder particles during an atomic layer coating (ALC) process;
each of the protein powder particles further comprises a sugar;
the sugar comprises sucrose, mannitol, lactose, trehalose, sorbitol, isomers thereof, derivatives thereof, salts thereof, or any combination thereof;
each of the coated particles comprises about 0.01 wt % to about 15 wt % of the sugar;
the plurality of protein powder particles has an average particle size of about 0.1 μm to about 1,000 μm;
each of the coated particles comprises about 0.5 wt % to about 10 wt % of aluminum oxide; and
each of the coated particles comprises about 90 wt % to about 99.5 wt % of the protein powder particles comprising myoglobin.
